(19) United States
(12) Patent Application Publication
(10) Pub. No.: US 2026/0356792 A1
(43) Pub. Date: Nov. 12, 2026

(54) PROCESS FOR THE MANUFACTURE OF A PROPYLENE-DERIVED CHEMICAL OF INTEREST, IN PARTICULAR AN ACRYLIC ESTER, FROM RENEWABLY-SOURCED ETHANOL

(71) Applicant: BASF SE, Ludwigshafen am Rhein (DE)

(72) Inventors: Stefan WILLERSINN, Houston, TX (US); Alois KINDLER, Ludwigshafen am Rhein (DE); Christian WEINEL, Ludwigshafen am Rhein (DE); Daniel KECK, Ludwigshafen am Rhein (DE); Thomas MACKEWITZ, Ludwigshafen am Rhein (DE); Dieter RAHN, Ludwigshafen am Rhein (DE); Johannes Lazaros Friedrich ELLER, Ludwigshafen am Rhein (DE)

(21) Appl. No.: 19/123,922

(22) PCT Filed: Oct. 27, 2023
(86) PCT No.: PCT/EP2023/080088

§ 371 Date: Apr. 24, 2025

(30) Foreign Application Priority Data
Oct. 28, 2022 (EP) 22204408.3
Nov. 17, 2022 (EP) 22207980.8

(51) Int. Cl. C07C 69/54; C07C 1/24; C07C 2/08; C07C 6/04; C07C 11/04; C07C 11/06; C07C 11/08; C07C 29/14; C07C 31/12; C07C 31/125; C07C 45/45; C07C 45/50; C07C 47/02; C07C 51/25; C07C 57/04; C07C 67/08

(52) U.S. Cl. CPC C07C 69/54; C07C 1/24; C07C 2/08; C07C 6/04; C07C 11/04; C07C 11/06; C07C 11/08; C07C 29/14; C07C 31/12; C07C 31/125; C07C 45/45; C07C 45/50; C07C 47/02; C07C 51/252; C07C 57/04; C07C 67/08

(57) ABSTRACT

A process for the manufacture of an acrylic ester selected from n-butyl acrylate and 2-ethylhexyl acrylate comprises subjecting a feedstock comprising a renewably-sourced ethanol to dehydration to produce a renewably-sourced ethylene stream. The renewably-sourced ethylene stream is subjected to an olefin-interconversion to obtain renewably-sourced propylene; the olefin-interconversion comprising ethylene dimerization to obtain n-butenes; and metathesis reaction between n-butenes obtained according to (i) and ethylene to obtain propylene. The renewably-sourced propylene is subjected to a sequence of chemical conversions to obtain an acrylic ester selected from n-butyl acrylate and 2-ethylhexyl acrylate, the sequence of chemical conversions comprising α), β), δ) and ε) or α), γ), δ) and ε): α) hydroformylation of the propylene to produce n-butyraldehyde; β) hydrogenation reaction of the n-butyraldehyde obtained in α) to produce n-butanol; γ) condensation of the n-butyraldehyde obtained in α) to produce 2-ethyl-3-hydroxyhexanal and subjecting the 2-ethyl-3-hydroxyhexanal to a hydrogenation reaction to produce 2-ethylhexanol; δ) oxidation reaction of the propylene to produce acrylic acid; ε) esterification reaction of the acrylic acid with one of n-butanol obtained in β) or 2-ethylhexanol obtained in γ) to produce an acrylic ester. The process provides a reaction scheme for renewably-sourced n-butyl acrylate and 2-ethylhexyl acrylate.

### TECHNICAL BACKGROUND

The present invention relates to a process for the manufacture of propylene-derived chemicals, in particular an acrylic ester selected from n-butyl acrylate and 2-ethylhexyl acrylate, from renewably-sourced ethanol.

Ethylene is a cornerstone of the modern petrochemical industries. Important ethylene derivatives (at the end of their respective chains) include (meth)acrylic acid, (meth)acrylic esters, isononanols, ethylhexanol, and ethylene glycols. One of the problems faced by the manufacture of chemicals and intermediates from ethylene is that the starting raw materials are from fossil fuels, such as natural gas or crude oil, which are non-renewable feedstocks. Steam cracking, which employs petroleum fractions and natural gas liquids as feedstocks, is the dominant method for large-scale ethylene production worldwide.

Lower olefins, such as isobutylene or propylene, are of significant interest for industrial and chemical applications. Isobutylene, also known as isobutene or 2-methylpropene, is a hydrocarbon of significant interest that is widely used as an intermediate in the production of industrially important products, including para-xylene, jet fuel blendstocks, gasoline oxygenates, isooctane, methacrolein, methyl methacrylate, and butyl rubber. Propylene is a hydrocarbon of significant interest that is widely used as an intermediate in the production of acrylic acid. Historically, lower olefins have been obtained through the catalytic or steam cracking of fossil fuel feedstocks.

Applicants have realized that the production of ethylene and ethylene derivatives compounds would benefit from the replacement of at least a part of the carbonaceous raw materials of fossil origin by renewable resources, such as carbonaceous matter derived from biomass. Of particular interest is the ethanol feedstock which is produced from renewable resources. Such renewably-sourced ethanol, also referred to as “bioethanol” or “hydrous fuel alcohol” can be prepared in large quantities from organic waste or biomass via fermentation. The different feedstocks for producing ethanol may be sucrose-containing feedstocks, e.g., sugarcane, starchy materials, e.g., corn, starch, wheat, cassava, lignocellulosic biomass, e.g., switchgrass, and/or agricultural waste. The purification or isolation of bioethanol is frequently carried out by complicated, multistage distillation.

Even after the purification processes, the advantage of bioethanol is frequently decreased by small amounts of impurities which it contains. Bioethanol impurities may include oxygen-containing organics, for example other alcohols such as isopropanol, n-propanol, and isobutanol, and/or aldehydes such as acetaldehyde. Bioethanol impurities may further include sulfur-containing impurities, such as inorganic sulfur compounds dialkyl sulfides, dialkyl sulfoxides, alkyl mercaptans, 3-methylthio-1-propanol, and/or sulfur-containing amino acids.

It would be desirable to integrate renewably-sourced ethanol into existing processes designed for the conversion of fossil-derived ethylene or its intermediates. However, some of the impurities may interfere with the downstream processes which use bioethanol as feedstock and which generate chemical products, especially when some of the downstream steps are catalytic conversions.

If efforts are not made to remove at least some of these impurities, the yield of desired intermediate and final products and efficacy of the overall process may be diminished.

US 2008/0312485 discloses a method for continuously producing propylene by dehydrating ethanol obtained from biomass to obtain ethylene and reacting ethylene with n-butene in a metathesis reaction. The n-butene is made by dimerization of ethylene which is obtained from biomass-derived ethanol [0033] and [0061].

WO 2010/066830 discloses the transformation of bioethanol to ethylene. The bioethanol is produced by fermentation of carbohydrates or from synthesis gas made by gasification of biomass. The ethylene is subsequently dimerized or oligomerized to, e.g., 1-butene and/or 1-hexene. The dimeric or oligomeric alpha-olefins are transformed into internal olefins that are subsequently subjected to metathesis with ethylene.

WO 2011/085223 discloses an integrated process to prepare renewable hydrocarbons. The process includes dehydrating renewable isobutanol to form a mixture of linear butenes and isobutene and dehydrating renewable ethanol to ethylene. Subsequently the butene mixture and the ethylene are reacted to form one or more renewable C₃-C₁₆ olefins.

EP 3 067 340 A discloses a process comprising fermenting a renewable source of carbon for the production of a mixture of alcohols comprising ethanol, isopropanol and 1-butanol; joint dehydration of the alcohols to produce a mixture of olefins comprising chiefly ethylene, propylene and linear butenes, the linear butenes being a mixture of 1-butene and 2-butenes (cis-and trans-isomers), besides water and by-products; removal of water, oxygenated compounds and other by-products from the mixture of olefins, to generate a mixture of olefin comprising chiefly ethylene, propylene and linear butenes; and passing the mixture of olefins through an isomerization bed so that 1-butene is isomerized to 2-butene and subsequently passing the mixture of olefins comprising chiefly ethylene, propylene and 2-butenes through a metathesis bed, for reaction between ethylene and 2-butenes, generating additional propylene.

WO 2009/098268 discloses a process for the dehydration of an alcohol to make an olefin. The alcohol may be ethanol that can be obtained from carbohydrates. For this purpose a stream comprising the ethanol and an inert component is contacted with a catalyst to give ethylene. It is indicated that the ethylene can be used for dimerization to butene and then isomerization to isobutene, dimerization to 1-butene, which is isomerized to 2-butene and further converted by metathesis with ethylene to propylene, or conversion to ethylene oxide and glycol. Experimental details are provided only for ethanol dehydration. A similar process is disclosed in WO 2011/089235.

WO 2009/098269 discloses a process for conversion of ethanol that can be obtained from carbohydrates to propylene. The ethanol is dehydrated to ethylene which is reacted with olefins having four carbon atoms or more to give propylene. WO 2009/098267 discloses a similar process.

WO 2021/067294 discloses a process for simultaneously dehydrating, dimerizing and metathesizing a C₂-C₅, alcohol which can be from biobased processes in one reactor to produce a C₂-C₇ olefin.

WO 2009/070858 discloses an integrated process for the production of ethylene-butylene copolymers. The ethylene is obtained by dehydration of ethanol that is produced by the fermentation of sugars. One method of obtaining 1-butylene used for the polymerization is indicated to be dimerization of ethylene produced by dehydration of ethanol that is produced by the fermentation of sugars. No details as to the dimerization are given.

In embodiments, the invention seeks to advise a reaction scheme that provides renewably-sourced light olefins, such as ethylene and propylene, which partially or fully replace the light olefins output from a steam cracker. These light olefins are used as building blocks for producing a variety of chemicals of interest. It is desirable that the renewably-sourced light olefins can be blended or used interchangeably with a fossil-derived intermediate of the same chemical structure without necessitating adjustments in downstream processes. This includes that the starting olefins of all branches of the value chains, which historically have been served by the steam cracker output, can be supplied at the same time on a renewably-sourced basis. In this way, the greenhouse gases footprint and/or the carbon footprint for the production of a chemical of interest is at least reduced.

### DETAILED DESCRIPTION OF THE INVENTION

To this effect, the present invention relates to a process for the manufacture of a chemical of interest selected from propylene-derived chemicals, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion, to obtain renewably-sourced propylene; the olefin-interconversion comprising (i) and (ii):
   (I) ethelyne dimerization to obtain n-butenes;
   (ii) metathesis reaction between n-butenes obtained according to (i) and ethylene to obtain propylene; and
c) subjecting the renewably-sourced propylene to a chemical conversion or sequence of chemical conversions to obtain the chemical of interest.

In particular, the present invention relates to a process for the manufacture of an acrylic ester selected from n-butyl acrylate and 2-ethylhexyl acrylate, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion, to obtain renewably-sourced propylene; the olefin-interconversion comprising (i) and (ii):
   (i) ethylene dimerization to obtain n-butenes; and
   (ii) metathesis reaction between n-butenes obtained according to (i) and ethylene to obtain propylene; and
c) subjecting the renewably-sourced propylene to a sequence of chemical conversions to obtain an acrylic ester selected from n-butyl acrylate and 2-ethylhexyl acrylate, the sequence of chemical conversions comprising α), β), δ) and ε) or α), γ), δ) and ε):
α) hydroformylation of the propylene to produce n-butyraldehyde;
β) hydrogenation reaction of the n-butyraldehyde obtained in α) to produce n-butanol;
γ) condensation of the n-butyraldehyde obtained in α) to produce 2-ethyl-3-hydroxyhexanal and subjecting the 2-ethyl-3-hydroxyhexanal to a hydrogenation reaction to produce 2-ethylhexanol; δ) oxidation reaction of the propylene to produce acrylic acid; ε) esterification reaction of the acrylic acid with one of n-butanol obtained in β) or 2-ethylhexanol obtained in γ) to produce an acrylic ester.

The process of the invention is preferably a continuous process in the sense that at least the upstream steps of a reaction route leading to a chemical of interest, including step a) and steps b-(i) and b-(ii), are carried out continuously. In a still more preferred embodiment all steps of a reaction route leading to a chemical of interest are carried out continuously. This does not preclude the presence of buffer volumes between subsequent reaction steps in a reaction route.

The present invention is based on the idea of eliminating impurities that are inherently present in renewably-sourced ethanol during the ethylene manufacturing process itself. Hence, the renewably-sourced ethylene, or the renewably-sourced propylene produced therefrom, can be blended or used interchangeably with a fossil-derived intermediate of the same chemical structure without necessitating adjustments in downstream processes.

It is envisaged that the renewably-sourced olefins involved in the process according to the invention may be blended with complementary olefins from other sources. This can ensure the efficient utilization of downstream processes, e.g., for transitional periods when supply of renewably-sourced olefin is limited. These complementary olefins, including complementary ethylene, complementary propylene and complementary n-butenes, may be fossil-based, partially renewably-sourced or renewably-sourced by another production route.

Hence in an embodiment, the process comprises:
blending the renewably-sourced ethylene with complementary ethylene prior to step b), the complementary ethylene not being obtained from renewably-sourced ethanol in accordance with step a); and/or
blending the renewably-sourced propylene with complementary propylene prior to steps c) and d), the complementary propylene not being obtained from renewably-sourced ethanol in accordance with steps a) and b); and/or
blending the renewably-sourced n-butenes with complementary n-butenes prior to step b)-(ii), the complementary n-butenes not being obtained from renewably-sourced ethanol in accordance with steps a) and b)-(i).

Examples for complementary ethylenes are ethylenes obtained by steam cracking of fossil based feeds, like naphtha, natural gas or crude oil. Examples for complementary propylenes are propylenes obtained by steam cracking of fossil based feeds, like naphtha, natural gas or crude oil. Examples for complementary n-butenes are n-butenes obtained by steam cracking of fossil based feeds, like naphtha, natural gas or crude oil.

In another aspect, the invention also relates to a process for enhancing the environmental sustainability of an acrylic ester selected from n-butyl acrylate and 2-ethylhexyl acrylate by blending or replacing fossil-derived propylene with renewably-sourced propylene to obtain a sustainability-enhanced propylene and subjecting the sustainability-enhanced propylene to a sequence of chemical conversions to obtain the acrylic ester, wherein the renewably-sourced propylene is obtained by
a) subjecting a feedstock comprising a renewably-sourced ethanol to dehydration to produce the renewably-sourced ethylene stream; and
b) subjecting the renewably-sourced ethylene stream to an olefin-interconversion to obtain the renewably-sourced propylene; the olefin-interconversion comprising (i) and (ii):
   (i) ethylene-dimerization to obtain n-butenes; and
   (ii) metathesis reaction between n-butenes obtained according to (i) and ethylene to obtain propylene;
the sequence of chemical conversions comprising α), β), δ) and ε) or α), γ), δ) and ε):
α) hydroformylation of the propylene to produce n-butyraldehyde;
β) hydrogenation reaction of the n-butyraldehyde obtained in α) to produce n-butanol;
γ) condensation of the n-butyraldehyde obtained in a) to produce 2-ethyl-3-hydroxyhexanal and subjecting the 2-ethyl-3-hydroxyhexanal to a hydrogenation reaction to produce 2-ethylhexano;
δ) oxidation reaction of the propylene to produce acrylic acid;
ε) esterification reaction of the acrylic acid with one of n-butanol obtained in β) or 2-ethylhexanol obtained in γ) to produce an acrylic ester.

The key advantage of the process according to the present invention is that it can be easily integrated into an existing production site in which one or more chemicals of interest are manufactured based on a fossil feedstock, in particular naphtha. This means that fossil-based ethylene and propylene can be fully or partially substituted by respective renewably-sourced ethylene and propylene. Hereby, one obtains a respective chemical of interest, the carbon atoms of which are fully or partially based on a renewable-sourced carbon (so-called “green” carbon).

Further benefits occur from the reduction of carbon dioxide emissions. The chemical conversions involved in a reaction route leading to an individual chemical of interest are usually less than 100% selective. The yield losses manifest themselves in the generation of by-products that vary depending on the type of reaction involved. Oxidation reactions of a substrate to a desired product, for example, are almost invariably accompanied to a certain extent by an over-oxidation of the substrate to form carbon oxides, in particular carbon dioxide. By a full or partial replacement of fossil ethylene and propylene by their renewably-sourced counterparts, the fossil-based carbon dioxide emissions of the entire production site can be reduced because respective emissions resulting from yield losses along the value chain are at least partially based on green carbon. The resulting carbon dioxide emissions therefore do not contribute to the green house emission of the production site. For example, in the production of acrylic acid (as further described below) carbon dioxide is formed due to full oxidation of propylene. Using a renewably-sourced propylene as obtained by the process according to the present invention therefore prevents the formation of fossil-based carbon dioxide emissions resulting from such productions.

In addition, in non-oxidative reactions the various species present may undergo a host of side reactions, which generate color forming species, oligomers, and various decomposition products or the like. These are generally removed during work-up, e.g., by distillation, yielding light boiler and/or high boiler fractions in addition to the desired product. The light boiler or high boiler fractions are conventionally used for their calorific value, i.e. combusted as fuel, or exploited as hydrocarbon source, e.g. as steam cracker feed. It should be appreciated that full or partial replacement of fossil ethylene and propylene by their renewably-sourced counterparts at the beginning of the processing chain reduces the emission of fossil-based carbon dioxide resulting from the combustion of downstream side-products.

Hence, it is envisaged that direct and indirect benefits are associated with the process of the invention with regard to any chemical of interest that is manufactured via the process according to the present invention.

The expressions “renewable” or “renewably-sourced” in relation to a chemical compound are used synonymously and mean a chemical compound comprising a quantity of renewable carbon, i.e., having a reduced or no carbon content of fossil origin. Renewable carbon entails all carbon sources that avoid or substitute the use of any additional fossil carbon from the geosphere. Renewable carbon can come from the biosphere, atmosphere or technosphere—but not from the geosphere. Thus, the expression “renewable” or “renewably-sourced” includes, in particular, biomass-derived chemical compounds. It also includes compounds derived from waste such as polymer residues, or from waste streams of chemical production processes.

The expression “chemical of interest” collectively refers to any desired compound appearing in a value chain starting out from and including ethylene. Thus, the expression includes any intermediates and final products. In certain cases, a chemical compound can be an intermediate and final product at the same time. For example, n-butyl acrylate can be the final product of a value chain and yet can be an intermediate when it is further processed, if desired.

All patent and literature documents addressed in the following are incorporated herein by reference in their entirety.

Bioethanol is a preferred form of renewably-sourced ethanol, although the scope of the invention is not limited to the use of bioethanol.

In the present invention, bioethanol refers to the ethanol obtained from a biomass feedstock, such as plant or non-crop feedstock containing a carbon source that is convertible to ethanol, for example by microbial metabolism. Typical carbon source examples are starch, sugars like pentoses or hexoses, such as glucose, fructose, sucrose, xylose, arabinose, or degradation products of plants, hydrolysis products of cellulose or juice of sugar canes, beet and the like containing large amounts of the above components.

Biomass feedstock can originate from several sources. Bioethanol production may be based on food crop feedstocks such as corn and sugar cane, sugarcane bagasse, cassava (first generation biofeedstock).

Another source of biomass feedstock is lignocellulosic materials from agricultural crops (second-generation biofeedstock). Potential feedstocks include agricultural residue by-products such as rice, straw (such as wheat, oat and barley straw), rice husk, and corn stover. Biomass feedstock may also be waste material from the forest products industry (wood waste) and saw dust or produced on purpose as an ethanol crop. Switchgrass and napier grass may be used as on-purpose crops for conversion to ethanol.

The first-generation bioethanol is produced in four basic steps:
(1) Enzymatic saccharification or hydrolysis of starch into sugars
(2) Microbial fermentation of sugars
(3) Purification by distillation to give hydrous ethanol
(4) Dehydration (water removal) to produce anhydrous ethanol

Second-generation feedstocks are considered as renewable and sustainable carbon source. Pretreatment of this feedstock is an essential prerequisite before it is subjected to enzymatic hydrolysis, fermentation, distillation, and dehydration. Pretreatment involves milling and exposure to acid and heat to reduce the size of the plant fibers and hydrolyze a portion of the material to yield fermentable sugars. Saccharification utilizes enzymes to hydrolyze another portion to sugar. Finally, fermentation by bioengineered microorganisms converts the various sugars (pentoses and hexoses) to ethanol. The production of bioethanol is well-known and carried out on an industrial large scale.

Renewably-sourced ethanol can also be obtained from carbon-containing waste materials like waste products from the chemical industry, garbage and sewage sludge. The production of ethanol from waste materials can be done by gasification to syngas and catalytic conversion thereof the ethanol, see for example Recent Advances in Thermo-Chemical Conversion of Biomass, 2015, Pages 213-250, https://doi.org/10.1016/B978-0-444-63289-0.00008-9, and Nat Commun 11, 827 (2020), https://doi.org/10.1038/s41467-020-14672-8.

### Dehydration of Renewably-sourced Ethanol

As a first step, the invention involves the dehydration of renewably-sourced ethanol. The production of ethylene by catalytic dehydration of ethanol is a well-known process. The reaction is commonly carried out at 300 to 400 °C and moderate pressure in the presence of a catalyst. Catalytic effects are reviewed in Ind & Eng Chem Research, 52, 28, 9505-9514 (2013), Materials 6, 101-115 (2013) and ACS Omega, 2, 4287-4296 (2017). Examples for catalysts are activated alumina or silica, phosphoric acid impregnated on coke, heteropoly acids (HPA salts), silica-alumina, molecular sieves such as zeoliths of the ZSM-5 type or SAPO-11 type, other zeolites or modified zeolites of various molecular structures with zeoliths and HPA salts being preferred.

Ethanol dehydration is, for example described in WO 2009/098268, WO 2010/066830, WO 2009/070858 and the prior art discussed therein, WO 2011/085223 and the prior art discussed therein, U.S. Pat. Nos. 4,234,752, 4,396,789, 4,529,827 and WO 2004/078336.

The ethanol dehydration reaction is in general carried out in the vapor phase in contact with a heterogeneous catalyst bed using either fixed bed or fluidized bed reactors. For fixed bed reactors, the operation can be either isothermal (with external heating system) or adiabatic (in the presence of a heat carrying fluid). The feedstock is vaporized and heated to the desired reaction temperature; the temperature drops as the reaction proceeds in the reactor. Multiple reactor beds are usually used in series to maintain the temperature drop in each bed to a manageable range. The cooled effluent from each bed is further heated to bring it to the desired inlet temperature of the subsequent beds. Moreover, a portion of the water is recirculated along with fresh and unreacted ethanol. The presence of water helps in moderating the temperature decrease in each bed.

Prior to dehydration, the renewably-sourced ethanol feedstock may be sent to a pretreatment section to remove mineral contaminants, which would otherwise be detrimental to the downstream catalytic reaction. The pretreatment may involve contacting the renewably-sourced ethanol feedstock with cation and/or anion exchange resins. After a certain period of operation, the resins may be regenerated by passing a regenerant solution through the resin bed(s) to restore their ion exchange capacity. Two sets of beds are preferably operated in parallel to maintain continuous operation. One set of resin beds is suitably regenerated while the other set is being used for pretreatment.

In the isothermal design, the catalyst is placed inside the tubes of multitubular fixed-bed reactors which arranged vertically and surrounded by a shell (tube and shell design). A heat transfer medium, such as molten salts or oil, is circulated inside the shell to provide the required heat. Baffles may be provided on the shell side to facilitate heat transfer. The cooled heating medium is heated externally and is recirculated. The temperature drop on the process side can be reduced as compared to the adiabatic reactor. A better control on the temperature results in increased selectivity for the ethylene formation and reduction in the amount of undesireable by-products. The temperature is maintained at approximately constant levels within the range of 300° to 350 °C. Ethanol conversion is between 98 and 99%, and the selectivity to ethylene is between 94 and 97 mol %. Because of the rate of coke deposition, the catalyst must be regenerated frequently. Depending on the type of catalyst used, the cycle life is between 3 weeks and 4 months, followed by regeneration, for example for 3 days.

In the adiabatic design, the endothermic heat of reaction is supplied by a preheated inert diluent such as steam. Three fixed-bed reactors may typically be used, with intermediate furnaces to reheat the ethanol/steam mixed feed stream to each reactor. Feeding steam with ethanol results in less coke formation, longer catalyst activity, and higher yields.

A further process is a fluidized-bed process. The fluidized-bed system offers excellent temperature control in the reactor, thereby minimizing by-product formation. The heat distribution rate of the fluidized bed operation approaches isothermal conditions. The endothermic heat of reaction is supplied by the hot recycled silica-alumina catalyst returning from the catalyst regenerator. Thus, external heating of the reactor is not necessary.

After dehydration, the reaction mixture is subjected to a separation step. The general separation scheme consists of quickly cooling the reaction gas, for example in a water quench tower, which separates most of the by-product water and the unreacted ethanol from ethylene and other light components which, for example exit from the top of the quench tower. In one type of separation scheme, the water-washed ethylene stream is immediately caustic-washed, for example in a column, to remove traces of CO₂. The gaseous stream may enter a compressor directly or pass to a surge gas holder first and then to a gas compressor. After compression, the gas is cooled with refrigeration and then passed through an adsorber with, for example activated carbon, to remove traces of heavy components, (e.g., C4s), if they are present. The adsorber is followed by a desiccant drying and dust filtering step before the ethylene product leaves the plant. This separation scheme produces 99%+purity ethylene. If desired, the ethylene is further purified by caustic washing and desiccant-drying, and fractionated in a low-temperature column to obtain the final product.

Several commercial processes are currently in operation, developed by Braskem, Chematur, British Petroleum (BP), and Axens together with Total and IFPEN. The processes differ, e.g., in their process conditions, catalysts and adopted heat integration scheme. The process by BP (now Technip) is called Hummingbird. In this process, a heteropoly acid is used as catalyst, and the reactor operates at 160 to 270 °C and 1 to 45 bar. The unreacted ethanol in recirculated to the reactor. The process developed by Axens is called Atol. Two fixed bed adiabatic reactors, operating at 400 to 500 °C, are used. Chematur's process operates with four adiabatic tubular reactors. Syndol catalysts, with the main components of Al₂O₃-MgO/SiO₂, are employed in this process that was developed by American Halcon Scientific Design, Inc. in the 1980s. In the Braskem process, the adiabatic reactor feed is diluted with steam to a large extent. In such a process, the reactor operates at 180 to 600 °C , preferably 300 to 500 °C , and at 1.9 to 19.6 bar. An alumina or silica-alumina catalyst is used. The Braskem process is described in more detail in U.S. Pat. No. 4,232,179. A process control in accordance with the Braskem process is particularly preferred.

### Dimerization of Ethylene

The process of the invention involves an ethylene-dimerization to obtain n-butenes in accordance with step b)-(i). Any known method can be used for ethylene dimerization to produce n-butenes. A review on dimerization and oligomerization chemistry and technology is given in Catalysis Today, vol. 14(no. 1), Apr. 10, 1992.

Expediently, step B)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream; and
fractionating the effluent to recover a stream consisting essentially of n-butenes, a stream consisting essentially of heavier olefins, and an optional ethylene stream.

The dimerization catalyst may be homogeneous or heterogeneous. Typical dimerization catalysts are titanium or nickel compounds activated with alkyl aluminium compounds. In general, the Ti(IV) valency is stabilized by selecting the appropriate ligands, alkyl aluminium compound, the solvent polarity and the Al/Ti ratio. Nickel compounds that can catalyse the selective production of butenes are typically based on cationic nickel salts stabilised with phosphine and activated with alkyl aluminium compounds.

In one embodiment, the oligomerization of ethylene is implemented in the presence of a catalytic system in the liquid phase comprising a nickel compound and an aluminum compound. Such catalytic systems are described in the documents FR 2 443 877 and FR 2794 038. The Dimersol ETM process is based on this technology and leads to the industrial production of olefins.

Thus, in one embodiment, the oligomerization of ethylene is implemented in the presence of a catalytic system comprising:
i) at least one bivalent nickel compound,
ii) at least one hydrocarbyl aluminum dihalide of formula AIRX₂, in which R is a hydrocarbyl radical comprising 1 to 12 carbon atoms, such as alkyl, aryl, aralkyl, alkaryl or cycloalkyl, X is a chlorine or bromine atom, and
iii) optionally a Brønsted organic acid.

As the bivalent nickel compound, nickel carboxylates of general formula (R¹COO)₂Ni are preferably used, where R¹ is an optionally substituted hydrocarbyl radical, for example alkyl, cycloalkyl, alkenyl, aryl, aralkyl, or alkaryl, containing up to 20 carbon atoms, preferably a hydrocarbyl radical of 5 to 20 carbon atoms, preferably 6 to 18 carbon atoms. Suitable bivalent nickel compounds include: chloride, bromide, carboxylates such as octoate, 2-ethylhexanoate, decanoate, oleate, salicylate, hydroxydecanoate, stearate, phenates, naphthenates, and acetyl acetonates. Nickel 2-ethylhexanoate is preferably used.

The hydrocarbyl aluminum dihalide compound corresponds to the formula AIRX₂, in which R is a hydrocarbyl radical comprising 1 to 12 carbon atoms, such as alkyl, aryl, aralkyl, alkaryl or cycloalkyl, and X is a chlorine or bromine atom. As examples of such compounds, it is possible to mention ethylaluminum sesquichloride, dichloroethyl aluminum, dichloroisobutyl aluminum, chlorodiethyl aluminum or mixtures thereof.

According to a preferred method, a Brønsted organic acid is used. The Brønsted acid compound corresponds to the formula HY, where Y is an organic anion, for example carboxylic, sulfonic or phenolic. Halocarboxylic acids of formula R²COOH in which R² is a halogenated alkyl radical are preferred, in particular those that contain at least one alpha-halogen atom of the group —COOH with 2 to 10 carbon atoms in all. Preferably, a haloacetic acid of formula CXₚH₃₋ₚ—COOH is used, in which X is fluorine, chlorine, bromine or iodine, with p being an integer from 1 to 3. By way of example, it is possible to cite the trifluoroacetic, difluoroacetic, fluoroacetic, trichloroacetic, dichloroacetic, and chloroacetic acids. It is also possible to use arylsulfonic, alkylsulfonic, and fluoroalkylsulfonic acids, and picric acid and nitroacetic acid. Trifluoroacetic acid is preferably used.

The three components of the catalytic formula can be mixed in any order. However, it is preferable first to mix the nickel compound with the Brønsted organic acid, and then next to introduce the aluminum compound. The molar ratio of the hydrocarbyl aluminum dihalide to the nickel compound, expressed by the Al/Ni ratio, is 2/1 to 50/1, and preferably 2/1 to 20/1. The molar ratio of the Brønsted acid to the nickel compound is 0.25/1 to 10/1, and preferably 0.25/1 to 5/1.

According to a preferred method, the hydrocarbyl aluminum dihalide can be enriched with an aluminum trihalide, the mixture of the two compounds then corresponding to the formula AIRₙX₃₋ₙ, in which R is a hydrocarbyl radical comprising 1 to 12 carbon atoms, such as alkyl, aryl, aralkyl, alkaryl or cycloalkyl, X is a chlorine or bromine atom, and n is a number between 0 and 1. Suitable mixtures include: dichloroethyl aluminum enriched with aluminum chloride, the mixture having a formula AlEt_{0.9}Cl_{2.1}; dichloroisobutyl aluminum enriched with aluminum chloride, the mixture having a formula AliBu_{0.9}Cl_{2.1}; and dibromoethyl aluminum enriched with aluminum bromide, the mixture having a formula AlEt_{0.9}Br_{2.1}.

The reaction for oligomerization of ethylene can be implemented at a temperature of −20 to 80 °C , preferably 40 to 60 °C , under pressure conditions such that the reagents are kept at least for the most part in the liquid phase or in the condensed phase. The pressure is generally between 0.5 and 5 MPa, preferably between 0.5 MPa and 3.5 MPa. The time of contact is generally between 0.5 and 20 hours, preferably between 1 and 15 hours.

The oligomerization stage can be implemented in a reactor with one or more reaction stages in a series, with the ethylene feedstock and/or the catalytic composition that is preferably pre-conditioned in advance being introduced continuously, either in the first stage, or in the first stage and any other one of the stages. At the outlet of the reactor, the catalyst can be deactivated, for example by injection of ammonia and/or an aqueous solution of soda and/or an aqueous solution of sulfuric acid. The unconverted olefins and alkanes that are optionally present in the feedstock are then separated from the oligomers by a separation stage, for example by distillation or washing cycles by means of caustic soda and/or water.

The conversion per pass is generally 85 to 98%. The selectivity of n-butenes that are formed is generally between 50 and 80%. The n-butenes consist of butene-2 (cis- and trans-) and butene-1.

The effluent generally contains less than 0.2% by weight of isobutene, or even less than 0.1% by weight of isobutene.

### Separation of a Stream Rich in n-Butenes

The effluent that is obtained by dimerization of ethylene is subjected to a separation stage in such a way as to obtain an n-butene-enriched fraction.

The separation can be carried out by evaporation, distillation, extractive distillation, extraction by solvent or else by a combination of these techniques. These processes are known by one skilled in the art. Preferably, a separation of the effluent that is obtained by oligomerization of ethylene is carried out by distillation.

Preferably, the effluent of the oligomerization is sent into a distillation column system comprising one or more columns that makes it possible to separate, on the one hand, n-butenes from ethylene, which can be returned to the oligomerization reactor, and heavier olefins with 5 carbon atoms and more.

The higher olefins may be subjected to hydrogenation so as to obtain renewably-sourced naphtha. “Renewably-sourced naphtha” shall mean naphtha produced from renewable sources. It is a hydrocarbon composition, consisting of mainly paraffins.

The molecular weight of this renewably-sourced naphtha may range from hydrocarbons having 5 to 8 carbon atoms. Renewably-sourced naphtha can be used as a feedstock in steam-cracking to produce renewably-sourced light olefins, dienes and aromatics.

Hence, in an embodiment, step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractionating the effluent to recover a stream consisting essentially of n-butenes, a stream consisting essentially of heavier olefins, and an optional ethylene stream; and
optionally subjecting the stream consisting essentially of heavier olefins to hydrogenation so as to obtain renewably-sourced naphtha.

### Metathesis of Ethylene With n-Butenes

Ethylene is able to undergo metathesis with n-butenes to produce propylene. Step b)-(ii) comprises a metathesis reaction between n-butenes obtained according to step (i) and ethylene to obtain propylene. The n-butenes obtained according during ethylene dimerization (i) are a mixed stream including 1-butene and 2-butenes. Essentially only the 2-butenes react in a metathesis reaction, while 1-butene is essentially inert.

In one embodiment, 1-butene is removed from the mixed stream of 1-butene and 2-butenes and directed to a use elsewhere in the plant. Thus, in one embodiment, step b)-(ii) comprises removal of 1-butene from the mixed stream to obtain a stream rich in 2-butenes, and subjecting the stream rich in 2-butenes to the metathesis reaction. A stream rich in 2-butenes may comprise at least 90 wt.-% of 2-butenes, based on the total amount of n-butenes.

Alternatively, 1-butene may be converted to 2-butene by double bond isomerization. Double bond isomerization is an equilibrium-limited reaction. It is thus advantageous to subject the mixed stream of n-butenes to metathesis so as to react 2-butene with ethylene prior to double bond isomerization of 1-butene. Hence, in one embodiment the n-butenes are a mixed stream including 1-butene and 2-butenes, and b)-(ii) comprises
b)-(iia) subjecting the mixed stream to the metathesis reaction to obtain propylene and unreacted 1-butene;
b)-(iib) subjecting the unreacted 1-butene to double bond isomerization to obtain 2-butenes; and
b)-(iic)recycling the 2-butenes obtained in step b)-(iib) to step b)-(iia).

In another embodiment, it is possible to convert 1-butene to 2-butene simultaneously with the metathesis reaction. For this purpose, a metathesis catalyst and an isomerization catalyst may be physically mixed or provided as distinct layers to allow both reactions to proceed simultaneously. Thus, in one embodiment, step b)-(ii) is carried out by passing the mixed stream through a metathesis/isomerization zone comprising both a metathesis catalyst and an isomerization catalyst. As 2-butene is consumed due to the metathesis reaction over the metathesis catalyst, it is thus replenished by isomerization of 1-butene to 2-butene over the isomerization catalyst.

The reaction is carried out in the presence of a metathesis catalyst on the basis of a metal which is selected from tungsten, molybdenum, rhenium, niobium, tantalum, vanadium, ruthenium, rhodium, iridium, osmium and nickel and the like. Tungsten, molybdenum and rhenium are preferred and tungsten is particularly preferred. Typically, tungsten catalysts are supported on silica, molybdenum and rhenium are supported on alumina based carriers. Especially preferred metathesis catalysts are WO₃-based catalysts, for example silica-supported WO₃ in the form of granules.

Suitable isomerization catalysts include magnesium-based catalysts such as MgO-based catalysts, for example tableted MgO.

Metathesis is carried out under conditions effective to produce an effluent comprising propylene, unconverted ethylene, and optionally 1-butene.

Unconverted ethylene and/or unconverted n-butenes may be recycled and combined with fresh ethylene and n-butenes to provided the metathesis feedstock.

The reaction may be conducted at 340-375 °C, 25-40 bar, a weight hourly space velocity (WHSV) of 7.5-30 hr⁻¹, and an ethylene to 2-butene molar ratio of 3:1 to 10:1.

The reactor effluent may be sent to a deethenizer to remove C2 and lighter material. The bottoms from the deethenizer are sent to the depropenizer. High-purity, polymer-grade propylene (>99.9% molar purity) is recovered from the depropenizer overhead. The lighter material from the deethenizer and heavier C₄₊ material from the depropenizer are partly recycled to the reactors. Purge streams are provided for the lighter and heavier material to prevent buildup of inerts.

It should be noted that propane is not produced during the metathesis reaction. Consequently, polymer-grade propylene can be produced from the process, without the need for an expensive propylene-propane superfractionator.

Commercial processes for producing polymer-grade propylene by metathesis from ethylene and butenes feedstock are available from CB&I/Lummus (tradnemame OCT™) and from LyondellBasell.

### Hydroformylation of Propylene

Hydroformylation of propylene produces n-butyraldehyde, isobutyraldehyde or a mixture thereof. If desired, the produced aldehydes can be separated by fractionation.

Hydroformylation or the oxo process is an important large-scale industrial process for preparing aldehydes from olefins, carbon monoxide and hydrogen. These aldehydes can optionally be hydrogenated with hydrogen in the same operation or subsequently in a separate hydrogenation step, to produce the corresponding alcohols. In general, hydroformylation is carried out in the presence of catalysts which are homogeneously dissolved in the reaction medium. Catalysts used are generally the carbonyl complexes of metals of transition group VIII, in particular Co, Rh, Ir, Pd, Pt or Ru, which may be unmodified or modified with, for example, amine-containing or phosphine-containing ligands. A summarizing account of the processes practiced on a large scale in industry is found in J. Falbe, “New Syntheses with Carbon Monoxide”, Springer Verlag 1980, p. 162 ff., U.S. Pat. Nos. 3,527,809; 3,917,661; 4,148,830; 4,742,178, 4,769,984; 4,885,401; 6,049,011.

Propylene is preferably hydroformylated using ligand-modified rhodium carbonyls as the catalyst. Hydroformylation of propylene can be carried out at temperatures in the range of 50 °C to 200 °C , preferably 60 °C to 150 °C , and more preferably 70 °C to 120 °C.

In one embodiment, the hydroformylation reaction is conducted at a low pressure, e.g., a pressure in the range of 0.05 to 50 MPa (absolute), and preferably in the range of about 0.1 MPa to 30 MPa, most preferably at a pressure below 5 MPa. Desirably, the partial pressure of carbon monoxide is not greater than 50% of the total pressure.

The proportions of carbon monoxide, hydrogen, and propylene in the hydroformylation reaction medium can be selected within a wide range. In some embodiments, based on the total amount of CO, hydrogen, and propylene, CO is from about 1 to 50 mol-%, preferably about 1 to 35 mol-%; H₂ is from about 1 to 98 mol-%, preferably about 10 to 90 mol-%; and propylene is from about 0.1 to 35 mol-%, preferably about 1 to 35 mol-%.

The hydroformylation reaction preferably takes place in the presence of both liquid and gas phases. The reactants generally are in the gas phase. The catalyst typically is in the liquid phase. Because the reactants are gaseous compounds, a high contact surface area between the gas and liquid phases is desirable to enhance good mass transfer. A high contact surface area between the catalyst solution and the gas phase may be provided in any suitable manner. In a batch process, the batch contents are thoroughly mixed during the course of the reaction. In a continuous operation the reactor feed gas can be contacted with the catalyst solution in, for example, a continuous-flow stirred autoclave where the gas is introduced and dispersed at the bottom of the vessel, preferably through a perforated inlet (e.g., a sparger). High contact between the catalyst and the gas feed may also be provided by dispersing the solution of the Rh catalyst on a high surface area support, a technique well known in the art as supported liquid phase catalysis, or providing the Rh as part of a permeable gel.

The reaction may be conducted either in a batch mode or, preferably, on a continuous basis. One or more reactors may be used in continuous modes to carry out the reaction in one or more stages.

The ratio of H₂ to CO in the syngas used for hydroformylation is desirably in the range from 1.1:1 to 1.01:1, preferably 1.06:1 to 1.02:1. Often, syngas may be made or otherwise initially provided in a manner such that the ratio of hydrogen to CO is much higher than this. The excess hydrogen can be separated and used in other reaction stages as desired. For example, the excess hydrogen may be used to reduce n-butyraldehyde to n-butanol. In some modes of practice, syngas in the practice of the present invention is anhydrous.

### Hydrogenation of n-Butyraldehyde

Hydrogenation of n-butyraldehyde, isobutyraldehyde or a mixture thereof produces n-butanol, isobutanol or a mixture thereof.

These aldehydes can optionally be hydrogenated with hydrogen in the same reaction step, or subsequently in a separate hydrogenation step, to produce the corresponding alcohol. The hydrogenation of the n-butyraldehyde to n-butanol is a well-known reaction and can be conducted by any suitable known process.

In one embodiment, the hydrogenation is carried out with hydrogen in the liquid or gas phase in the presence of a hydrogenation catalyst. Homogeneous or heterogeneous catalysts can be used. Copper catalysts have proved to be the most suitable. Typically, the reaction is carried out in the liquid phase on fixed-bed catalysts at 20 to 200 °C and pressures of up to 30 MPa. Hydrogenation in the gas phase is preferably carried out continuously. Further details can be taken from Ullmann's Encyclopedia of Industrial Chemistry, 5^{th} edition, vol. A1, 1984.

### Condensation of n-Butyraldehyde to Produce 2-Ethyl-3-Hydroxyhexanal

Step c) further comprises condensation of the n-butyraldehyde to produce 2-ethyl-3-hydroxyhexanal and, optionally, subjecting the 2-ethyl-3-hydroxyhexanal to a hydrogenation reaction to produce 2-ethylhexanol.

An aldol condensation is a well-known condensation reaction in which an enol or an enolate ion reacts with a carbonyl compound to form a β-hydroxyaldehyde or β-hydroxyketone (an aldol reaction) in the presence of an acid or base catalyst, followed by dehydration to give a conjugated enone and hydrogenation to the corresponding alcohol. In the present case n-butyraldehyde is reacted in a self-aldol condensation to obtain 2-ethyl-3-hydroxyhexanal.

Aldol condensations can occur under a variety of conditions under weak acidic or strong basic conditions and in the presence of various catalysts. The reaction can typically be carried out in liquid phase using an aqueous caustic catalyst at a temperature of about 80 to 140 °C. In another embodiment, the reaction can be carried out in gaseous phase by contacting the aldehyde in the vapor phase with a particulate catalyst comprising at least one basic alkali metal compound on an inert substrate at a temperature above 175 °C. Further details are provided in WO 2000/031011.

The obtained 2-ethyl-3-hydroxyhexanal can be hydrogenated to 2-ethylhexanol. The hydrogenation can be carried out analogously to the above-described hydrogenation of n-butyraldehyde and/or isobutyraldehyde.

### Oxidation of Propylene to Produce Acrolein or Acrylic Acid

Step c) comprises an oxidation reaction to produce an intermediate selected from acrolein and acrylic acid.

Acrylic acid is an important basic chemical. Owing to its very reactive double bond and the acid function, it is suitable in particular for use as monomer for preparing polymers. Of the amount of acrylic acid monomer produced, the major part is esterified before polymerization, for example to form acrylate adhesives, dispersions or coatings. Only the smaller part of the acrylic acid monomer produced is polymerized directly, for example to form water-absorbent resins. Whereas, in general, the direct polymerization of acrylic acid requires high purity monomer, the acrylic acid for conversion into acrylate before polymerization does not have to be so pure.

It is common knowledge that acrylic acid can be produced by heterogeneously catalyzed gas phase oxidation of propylene with molecular oxygen over solid catalysts at temperatures between 200° to 400 °C in two stages via acrolein (cf. for example DE-A 19 62 431, DE-A 29 43 707, DE-C 1 205 502, EP-A 257 565, EP-A 253 409, DE-B 22 51 364, EPA 117 146, GB-C 1 450 986 and EP-A 293 224). The catalysts used are oxidic multicomponent catalysts based for example on oxides of the elements molybdenum, chromium, vanadium or tellurium. Five most commonly used catalyst systems for acrolein production are cuprous oxides, uranium antimony oxides, tin antimony oxides, bismuth molybdate oxides and multi-component bismuth molybdate based oxides. The most efficient catalysts for partial oxidation of propylene to acrolein consist of multi-component metal oxides systems. In almost every multi-component catalyst system, bismuth molybdate serves as the main ingredient. The following components are most commonly used as catalyst additives in molybdate bismuth oxide based catalysts: iron, cobalt, nickel, tungsten, potassium and phosphorous. Typical catalyst supports are inert porous solids, such as SiO₂, Al₂O₃, MgO, TiO₂, ZrO₂, aluminosilicates, zeolites, activated carbon, and ceramics.

The oxidation of propylene to acrylic acid can be carried out in one stage or two stages. Catalysts used for the heterogeneously catalyzed reaction are as a rule multimetal oxide materials which generally contain heavy metal molybdates as main component and compounds of various elements as promoters. The oxidation of propylene takes place in a first step to give acrolein and in a second step to give acrylic acid. Since the two oxidation steps may differ in their kinetics, uniform process conditions and a single catalyst do not as a rule lead to optimum selectivity. Recently, two-stage processes with optimum adaptation of catalyst and process variables have therefore preferably been developed. In general, propylene is oxidized to acrolein in the presence of molecular oxygen in the first stage in an exothermic reaction in a fixed-bed tubular reactor. The reaction products are passed directly into the second reactor and are further oxidized to acrylic acid. The reaction gases obtained in the second stage can be condensed and the acrylic acid can be isolated therefrom by extraction and/or distillation.

The oxidation of propylene to acrolein and/or acrylic acid is highly exothermic. The tubes of the fixed-bed tubular reactor which are filled with the heterogeneous catalyst are therefore surrounded by a cooling medium, as a rule a salt melt, such as a eutectic mixture of KNO₃ and NaNO₂. The heat of reaction is released through the wall of the catalyst-filled tubes to the salt bath.

Particularly preferred multimetal oxide materials have the formula I or II

[X¹ₐX²_{b}, Oₓ]ₚ[X³_{c}X⁴_{d}X⁵ₑX⁶_{f}X⁷_{g}X²ₕO_{y}]_{q} (I)

MO₁₂BiᵢX⁸ₖFeₗX⁹ₘX¹⁰ₙO_{z} (II)

where
X¹ is bismuth, tellurium, antimony, tin and/or copper, preferably bismuth,
X² is molybdenum and/or tungsten,
X³ is an alkali metal, thallium and/or samarium, preferably potassium,
X⁴ is an alkaline earth metal, nickel, cobalt, copper, manganese, zinc, tin, cadmium and/or mercury, preferably nickel and/or cobalt,
X⁵ is iron, chromium, cerium and/or vanadium, preferably iron,
X⁶ is phosphorus, arsenic, boron and/or antimony,
X⁷ is a rare earth metal, titanium, zirconium, niobium, tantalum, rhenium, ruthenium, rhodium, silver, gold, aluminum, gallium, indium, silicon, germanium, lead, thorium and/or uranium, preferably silicon, aluminum, titanium and/or zirconium,
a is from 0.01 to 8,
b is from 0.1 to 30,
c is from 0 to 4,
d is from 0 to 20,
e is from 0 to 20,
f is from 0 to 6,
g is from 0 to 15,
h is from 8 to 16,
x and y are numbers which are determined by the valency and frequency of the elements other than oxygen in I,
p and q are numbers whose ratio p/q is from 0.1 to 10,
X⁸ is cobalt and/or nickel, preferably cobalt,
X⁹ is silicon and/or aluminum, preferably silicon,
X¹⁰ is an alkali metal, preferably potassium, sodium, cesium and/or rubidium, in particular potassium,
i is from 0.1 to 2,
k is from 2 to 10,
l is from 0.5 to 10,
m is from 0 to 10,
n is from 0 to 0.5,
z is a number which is determined by the valency and frequency of the elements other than oxygen in II.

Multimetal oxide materials of the formula I are known per se from EP 0 000 835 and EP 0 575 897, and multimetal oxide materials of the formula II are known per se from DE 198 55 913.

Briefly, a process for preparing acrylic acid typically comprises the steps of:
(a) catalytic gas phase oxidation of propylene and/or acrolein to acrylic acid to obtain a gaseous reaction product comprising acrylic acid;
(b) solvent absorption of the reaction product;
(c) distillation of the solvent loaded with reaction product in a column to obtain a crude acrylic acid and the solvent, (d) purification of the crude acrylic acid by crystallization.

Step (a) affords not pure acrylic acid, but a gaseous mixture which in addition to acrylic acid can substantially include unconverted acrolein and/or propylene, water vapor, carbon monoxide, carbon dioxide, nitrogen, oxygen, acetic acid, propionic acid, formaldehyde, further aldehydes and maleic anhydride.

The remaining, unabsorbed reaction gas of step (a) is further cooled down so that the condensable part of the low-boiling co-components thereof, especially water, formaldehyde and acetic acid, may be separated off by condensation. This condensate is known as acid water. The remaining gas stream, hereinafter called recycle gas, consists predominantly of nitrogen, carbon oxides and unconverted starting materials. Preferably, the recycle gas is partly recirculated into the reaction stages as diluting gas.

The oxidation of propylene to acrolein, as well as the oxidation of acrolein to acrylic acid, proceed with less than 100% selectivity and are accompanied by the combustion of propylene or acrolein over the catalyst, which gives carbon monoxide and carbon dioxide, herein collectively referred to as COₓ. It should be appreciated that emission of the carbon dioxide side product does not contribute to the carbon footprint of this process, as the starting propylene is carbon neutral.

### Hydrogenation of COₓ From Oxidation Reaction

In an embodiment of this aspect, the propylene oxidation reaction progresses under formation of COₓ as a side product, and the process further comprises subjecting said COₓ to hydrogenation to produce at least one of synthesis gas, methanol, formaldehyde and formic acid. While the carbon monoxide and/or carbon dioxide may be sequestered by, e.g., underground storage, it may be beneficial to subject said COx to hydrogenation to produce at least one of synthesis gas, methanol, formaldehyde and formic acid.

The synthesis gas, methanol, formaldehyde and/or formic acid thus produced may then be certified as carbon negative, and can at least partly displace their fossil-based counterparts and reduce the carbon footprint of chemical conversion processes making use of synthesis gas, methanol, formaldehyde and/or formic acid.

### Esterification of Acrylic Acid to Produce an Acrylic Ester

Step c) further comprises an esterification reaction of the acrylic acid to produce an acrylic acid ester, in particular an acrylic acid ester selected from n-butyl acrylate and 2-ethylhexyl acrylate.

Acrylic esters are generally known and are important, for example, as reactive monoethylenically unsaturated monomers for the preparation of aqueous polymer dispersions by the free radical aqueous emulsion polymerization method, which dispersions are used, for example, as adhesives.

The acrylic acid can be esterified in a conventional manner to produce the desired acrylic acid ester using the corresponding alkanol such as methanol, ethanol, n-propanol, isopropanol, n-butanol or 2-ethylhexanol.

Processes for the preparation of alkyl acrylates by reacting acrylic acid with alkanols in the homogeneous liquid phase at elevated temperatures and in the presence of catalysts are equilibrium reactions in which the conversion of the acrylic acid and of the alkanol to the corresponding ester is limited by the equilibrium constant. Consequently, for an economical procedure, the unconverted starting materials have to be separated from the resulting ester and recycled to the reaction zone.

Conveniently, the reaction zone may consist of a cascade of reaction regions, connected in series, and the discharge stream of one reaction region forms a feed stream of a subsequent reaction region and the concentration of the esterification catalyst increases along the reaction cascade. Acrylic acid, the alkanol and the catalyst are fed continuously to the reaction zone. An azeotropic mixture comprising the alkyl acrylate, water and optionally starting alkanol is separated off by rectification via the top of a rectification zone mounted on the reaction zone. The azeotropic mixture is separated into an organic phase containing the alkyl acrylate and an aqueous phase, with a part of the organic phase being recycled to the reaction zone. The alkyl acrylate is isolated from the excess organic phase. The latter is usually carried out by separation steps involving rectification (cf. for example DE 19536178).

The temperature in the reaction zone depends on the type of alcohol used and is suitably in the range of 70 to 160 °C , preferably 100 to 140 °C. The total residence time of the reactants in the reaction zone is as a rule from 0.25 to 15 h, frequently from 1 to 7 h, or from 2 to 5 h.

Suitable acidic esterification catalysts include acidic ion exchange resins and strong mineral acids, e.g. sulfuric acid, or organic sulfonic acids, such as methanesulfonic acid, benzenesulfonic acid, dodecanesulfonic acid or para-toluenesulfonic acid, or a mixture of some or all of the abovementioned acids. Sulfuric acid is particularly suitable for carrying out the novel process. This applies in particular to the preparation of n-butyl acrylate.

The content of acidic esterification catalyst in the reaction zone is expediently from 0.1 to 20 wt.-%, frequently from 0.5 to 5 wt.-%, based on the reaction mixture contained therein.

To prevent undesired formation of polymer initiated by free radicals, a polymerization inhibitor is typically used during esterification. Examples of suitable polymerization inhibitors are hydroquinone, 4-methoxyphenol, and phenothiazine, which may be used singly or in admixture with each other. It is usual to add from about 0.01 to 0.1 wt.-% of polymerization inhibitor to the esterification mixture and mixtures containing the methacrylic ester.

### Additional Embodiments

The present invention also relates to processes for the manufacture of a chemical of interest selected from propylene-derived chemicals according to the following embodiments.
1. Process for the manufacture of a chemical of interest selected from propylene-derived chemicals, said process comprising the steps of:
   a) subjecting a feedstock comprising a renewably-sourced ethanol to dehydration to produce a renewably-sourced ethylene stream;
   b) subjecting the renewably-sourced ethylene stream to an olefin-interconversion, to obtain renewably-sourced propylene; the olefin-interconversion comprising (i) and (ii):
      (i) ethylene dimerization to obtain n-butenes;
      (ii) metathesis reaction between n-butenes obtained according to (i) and ethylene to obtain propylene; and
   c) subjecting the renewably-sourced propylene to a chemical conversion or sequence of chemical conversions to obtain the chemical of interest.
2. Process according to embodiment 1, wherein step c) comprises an oxidation reaction to produce an intermediate selected from acrolein and acrylic acid.
3. Process according to embodiment 2, wherein step c) further comprises an esterification reaction of the acrylic acid to produce an acrylic ester.
4. Process according to embodiment 2, wherein step c) further comprises polymerizing the acrylic acid, optionally together with one or more comonomers, to produce a water-absorbent resin.
5. Process according to embodiment 2, wherein step c) further comprises reacting the acrylic acid with isobutene to produce t-butyl acrylate.
6. Process according to embodiment 1, wherein step c) comprises hydroformylation of the renewably-sourced propylene to produce n-butyraldehyde, isobutyraldehyde or a mixture thereof.
7. Process according to embodiment 6, wherein step c) further comprises a hydrogenation reaction of the n-butyraldehyde, isobutyraldehyde or a mixture thereof to produce n-butanol, isobutanol or a mixture thereof.
8. Process according to embodiment 6, wherein step c) further comprises condensation of the n-butyraldehyde to produce 2-ethyl-3-hydroxyhexanal and, optionally, subjecting the 2-ethyl-3-hydroxyhexanal to a hydrogenation reaction to produce 2-ethylhexanol.
9. Process according to embodiment 7 or 8, wherein step c) further comprises esterification of the n-butanol, the isobutanol and/or the 2-ethylhexanol with (meth)acrylic acid to produce a (meth)acrylic ester.
10. Process according to embodiment 6, wherein step c) further comprises a condensation reaction of isobutyraldehyde with formaldehyde to produce hydroxypivaldehyde, and, optionally, subjecting the hydroxypivaldehyde to a hydrogenation reaction to produce neopentyl glycol.
11. Process according to embodiment 1, wherein step c) comprises an epoxidation reaction to produce propylene oxide, and
   optionally, a hydrolysis reaction of the propylene oxide to produce propylene glycols, or
   optionally, a ring-opening polymerization of the propylene oxide to produce polypropylene glycols.

### Polymerization of Acrylic Acid

In an embodiment, step c) further comprises polymerizing the acrylic acid, optionally together with one or more comonomers, to produce a water-absorbent resin.

Water-absorbent resins are used to produce diapers, tampons, sanitary napkins and other hygiene articles, but also as water-retaining agents in market gardening. The water-absorbent resins are also referred to as superabsorbents. The production of water-absorbent resins is described in the monograph “Modern Superabsorbent Polymer Technology”, F. L. Buchholz and A. T. Graham, Wiley-VCH, 1998, pages 71 to 103.

A typical process for producing water-absorbent resins comprises polymerizing a monomer solution or suspension comprising
a) acrylic acid which may be at least partly neutralized,
b) at least one crosslinker,
c) at least one initiator,
d) optionally an ethylenically unsaturated monomer copolymerizable with acrylic acid, and
e) optionally one or more water-soluble polymers,
drying the resulting polymer gel, grinding the dried polymer gel, classifying and thermally surface postcrosslinking.

Suitable crosslinkers b) are compounds having at least two groups suitable for crosslinking. Crosslinkers b) are preferably compounds having at least two polymerizable groups which can be polymerized by free-radical polymerization into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1 , di- and triacrylates, as described in EP 0 547 847 A1 , EP 0 559 476 A1 , EP 0 632 068 A1, WO 93/21237 A1, WO 03/104299 A1 , WO 03/104300 A1 , WO 03/104301 A1 and DE 103 31 450 A1, mixed acrylates which, as well as acrylate groups, comprise further ethylenically unsaturated groups, as described in DE 103 31 456 A1 and DE 103 55 401 A1 , or crosslinker mixtures, as described, for example, in DE 195 43 368 A1 , DE 196 46 484 A1, WO 90/15830 A1 and WO 02/032962 A2 . The amount of crosslinker b) is preferably 0.25 to 1.5 wt.-%, more preferably 0.3 to 1.2 wt.-% and most preferably 0.4 to 0.8 wt.-%, based on unneutralized acrylic acid. With rising crosslinker content, the centrifuge retention capacity falls and the absorption under pressure passes through a maximum.

The initiators c) used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite.

Suitable ethylenically unsaturated monomers d) copolymerizable with acrylic acid include acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

Suitable water-soluble polymers e) include polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Expediently, polymerization is carried out in a polymerization reactor or kneader having at least two shafts rotating in an axially parallel manner. Typically, an aqueous monomer solution is used. The water content of the monomer solution is preferably from 40 to 75 wt.-%, more preferably from 45 to 70 wt.-% and most preferably from 50 to 65 wt.-%.

The polymer gel is then preferably dried, e.g., with a belt drier, until the residual moisture content is preferably 0.5 to 15 wt.-%, more preferably 1 to 10 wt.-% and most preferably 2 to 8 wt.-%. Thereafter, the dried polymer gel is ground and classified. The apparatus used for grinding may typically be single or multistage roll mills, preferably two-stage or three-stage roll mills, pin mills, hammer mills or vibratory mills.

The mean particle size of the polymer particles removed as the product fraction is preferably at least 200 μm, more preferably from 250 to 600 μm and most preferably from 300 to 500 μm.

To further improve the properties, the polymer particles can be surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2 , di- or polyfunctional alcohols, as described in DE 33 14 019 A1 , DE 35 23 617 A1 and EP 0 450 922 A2 , or β-hydroxyalkylamides, as described in DE 102 04 938 A1 and U.S. Pat. No. 6,239,230. Preferred surface postcrosslinkers are ethylene carbonate, ethylene glycol diglycidyl ether, reaction products of polyamides with epichlorohydrin and mixtures of propylene glycol and 1,4-butanediol.

Reaction of Acrylic Acid With Isobutene to Produce t-Butyl Acrylate

In an embodiment, step c) further comprises reacting the acrylic acid with isobutene to produce t-butyl acrylate.

tert-Butyl acrylates are important starting materials for the preparation of polymers which are used, inter alia, as a constituent of paints, adhesives or coating resins. tert-Butyl esters of this kind are generally prepared by acid-catalyzed addition of a carboxylic acid onto isobutene (Houben-Weyl, Methoden der Organischen Chemie [Methods of Organic Chemistry], vol. 8, 1952, p. 534; U.S. Pat. Nos. 3,031,495 and U.S. 3,082,246). Catalysts used are acids soluble in the reaction mixture, for example mineral acids or alkyl-or arylsulfonic acids (DE-A-12 49 857, U.S. Pat. Nos. 3,087,962, 3,088,969), or insoluble catalysts such as acidic exchanger resins (U.S. Pat. Nos. 3,037,052, 3,031,495, DE-A-31 05 399, EP-A-268 999).

The reaction of acrylic acid with isobutene is generally effected in the absence of a solvent and in the liquid phase. Catalysts used are therefore those which are at least partly soluble in the reaction mixture. Suitable catalysts are strong inorganic or organic acids. Strong inorganic acids are, for example, mineral acids such as sulfuric acid, phosphoric acid and polyphosphoric acid, preferably sulfuric acid. Strong organic acids are, for example, sulfonic acids such as p-toluene-, benzene-, dodecylbenzene-and methanesulfonic acid, preferably p-toluenesulfonic acid and methanesulfonic acid. The inorganic catalysts in particular are only partly soluble in the reaction mixture on commencement of the reaction. In the course of the reaction, the solubility of the catalyst improves (primarily because of the formation of a partial ester of the catalyst, for example the sulfuric monoester). At least in the last section, it is therefore generally in solution in the reaction mixture.

The concentration of the catalyst in the reaction mixture is generally about 0.1% to 10% by weight, preferably 0.5% to 5% by weight, based on the total amount of the reaction mixture.

The reaction of the acrylic acid with isobutene in the presence of an acidic catalyst is effected preferably in conventional reaction vessels or in columns (DE-A-11 28 428). A suitable reactor is described by way of example in WO 02/10109 A1 . Preferably, the reaction is conducted in a reactor, which is especially a cylindrical reactor. The reactor is divided into a plurality of, preferably 3, 4 or 5, separate sections. The volume of the reactor sections may be the same or different. Preferably, the volume of the first reactor section is greater than that of the remaining sections.

The resulting reaction mixture is withdrawn at the upper end of the reactor and sent to further workup. Unconverted gaseous isobutene accumulates in the upper region of the reactor. Preferably, condensable organic compounds, such as unconverted acrylic acid, are condensed out of the isobutene-containing gas stream taken off at the upper end of the reactor and thus are freed of gases that are inert with respect to the reaction, such as air and butane. Unconverted isobutene dissolves partly in the condensed constituents. The condensed organic compounds may then be fed back into the reaction in liquid form.

The reaction temperature is typically in the range from about 10 to 40 °C. It is preferably controlled in such a way that it is at its highest in the first reactor section. Preferably, the reaction temperature in the first reactor section is in the range from about 30 to 40 °C. It is lower in the second section, preferably by about 5 to 15 °C. The temperature in the sections that follow downstream of the second section may be the same or different. It is generally not higher than in the second section, preferably lower, especially by about 3 to 10 °C. In the fourth section, it is generally as high as in the third section or about 1 to 5 °C lower. The temperature in the last reactor section is preferably in the range from about 10 to 25 °C.

The reaction can be conducted at reduced pressure, ambient pressure or slightly elevated pressure (100 to 300 mbar abs.), or preferably at elevated pressure (e.g. 0.5 to 3 bar).

### Esterification of n-Butanol, Isobutanol and/or 2-Ethylhexanol

In an embodiment, step c) further comprises esterification of the n-butanol, the isobutanol and/or the 2-ethylhexanol with carboxylic acids. Suitable carboxylic acids include saturated and non-saturated C₁-C₁₆-carboxylic acid, in particular (meth) acrylic acid, as described above with regard to the esterification of acrylic acid to produce a acrylic ester. When the carboxylic acid is a saturated carboxylic acid, a polymerization inhibitor is not required.

Esters of C₄-C₁₀ carboxylic acids, such as phthalic acid and adipic acid, and n-butanol and/or 2-ethylhexanol are widely used as plasticizers in plastics, such as cellulose acetates, polyurethanes, PVC, polyacrylates, etc. They may be prepared by reacting the acid component or an anhydride thereof with the alcohol component in the presence of an esterification catalyst. The reaction is an equilibrium reaction. The equilibrium may be shifted to the product side, i.e. the ester side, by continuous removal of the water produced as by-product from the reaction. 2-Ethylhexanol has a region in which it is not miscible with water; hence it is possible to distill off continuously from the reaction mixture a mixture of the water of the reaction and 2-ethylhexanol, and, after phase separation, to return the organic phase to the esterification, while the aqueous phase is removed from the system.

### Condensation of Isobutyraldehyde With Formaldehyde

In an embodiment, step c) further comprises a condensation reaction of isobutyraldehyde with formaldehyde, i.e., a cross-aldol condensation, to produce hydroxypivaldehyde.

An aldol condensation is a well-known condensation reaction in which an enol or an enolate ion reacts with a carbonyl compound to form a β-hydroxyaldehyde or β-hydroxyketone (an aldol reaction) in the presence of an acid or base catalyst, followed by dehydration to give a conjugated enone and hydrogenation to the corresponding alcohol. In the present case isobutyraldehyde is reacted with formaldehyde in a cross-aldol condensation to obtain hydroxypivaldehyde.

Aldol condensations can occur under a variety of conditions under weak acidic or strong basic conditions and in the presence of various catalysts. In the present case a catalyst system comprising a secondary amine, preferably a di-C₁-C₆-alkylamine or a 4- , 5- or 6-membered cyclic mono or diamine, and an organic acid with up to 10 carbon atoms, preferably a mono or dicarboxylic acid, is preferably used. Examples for amines are dimethylamine, diethylamine, methyl ethyl amine, ethyl butyl amine, di-n-butylamine, di-2-ethylhexylamine, diisooctylamine, diphenylamine, dicyclohexylamine, piperidine, piperazine or morpholine or a combination thereof. Examples for organic acids are formic, acetic, propionic, malic, malonic, glutaric, tartaric, adipic, succinic, hydroxy succinic, maleic, 2-ethylhexanoic or salicylic acid or combinations thereof.

The molar ratio of formaldehyde to isobutyraldehyde is preferably in the range of 1:1 to 1.5:1. The secondary amine is preferably used in a molar ratio of 0.005:1 to 0.1:1 to the propionaldehyde. The organic acid may be used in a molar ratio of 0.002:1 to 0.05:1 to propionaldehyde.

The reaction temperature may be in the range from 70 °C to 120 °C , preferably 80 °C to 100 °C , and the pressure may be from 100 kPa to 300 kPa, preferably 150 to 250 kPa.

The produced hydroxypivaldehyde may be subjected to a hydrogenation reaction to produce neopentyl glycol. The hydrogenation can be carried out analogously to the above-described hydrogenation of n-butyraldehyde and/or isobutyraldehyde.

### Epoxidation of Propylene

In another aspect of the invention, the renewably-sourced C₃₋₄-olefin is propylene, and step c) comprises an epoxidation reaction to produce propylene oxide, and
optionally, a hydrolysis reaction of the propylene oxide to produce propylene glycols, or
optionally, a ring-opening polymerization of the propylene oxide to produce polypropylene glycols.

The oxidation of propylene is typically carried out with an organic peroxide. The following hydroperoxides are generally used:
1) t-Butyl hydroperoxides, derived from the oxygenation of isobutane (Halcon process).
2) Ethylbenzene hydroperoxide, derived from the oxygenation of ethylbenzene.
3) Cumene hydroperoxides, derived from the oxygenation of cumene (isopropylbenzene).
4) Hydrogen peroxide, or a hydrogen peroxide source, catalyzed by a titanium-doped silicalite (HPPO process).

Preferably, the oxidation of propylene is carried out using hydrogen peroxide, or a hydrogen peroxide source. Using hydrogen peroxide or a hydrogen peroxide source has the advantage that water is obtained as a side product, rather than an alcohol.

In one embodiment, the oxidation of propylene comprises
1) introducing a feed stream comprising propylene, hydrogen peroxide or a hydrogen peroxide source, and an organic solvent into a reactor containing a catalyst;
2) subjecting the feed stream to epoxidation conditions in the presence of the catalyst so as to obtain a reaction mixture comprising propylene oxide and the organic solvent;
3) removing a product stream comprising the propylene oxide and the organic solvent from the reactor.

In case that hydrogen peroxide is employed, it is preferred that the hydrogen peroxide is an aqueous hydrogen peroxide solution, wherein the solution comprises preferably 30 to 50 wt.-% hydrogen peroxide relative to the total amount of water.

It is also possible that the hydrogen peroxide is formed in situ in the reaction mixture from hydrogen and oxygen in the presence of a suitable catalyst or catalyst system, for example in the presence of a titanium containing zeolite additionally containing one or more noble metals, or a titanium containing zeolite and an additional catalyst containing one or more noble metals, for example supported on a suitable support such as charcoal or a suitable inorganic oxide or mixture of inorganic oxides.

Suitable organic solvents include alcohols, nitriles, and mixtures thereof, optionally also water. Preferably, the organic solvent is selected methanol and acetonitrile. Most preferably, the organic solvent is acetonitrile.

Generally, the feed stream is not restricted regarding the molar ratio of propylene and hydrogen peroxide or one equivalent of hydrogen peroxide resulting from the hydrogen peroxide source. Preferably, propylene is present in a molar excess in the feed stream with regard to hydrogen peroxide or one equivalent of hydrogen peroxide resulting from the hydrogen peroxide source. Preferably, the molar ratio of propylene and hydrogen peroxide or one equivalent of hydrogen peroxide resulting from the hydrogen peroxide source in the feed stream is from 1 to 1.6, more preferably from 1.1 to 1.55, more preferably from 1.2 to 1.5, more preferably from 1.40 to 1.45.

Typically, the catalyst is a titanium containing zeolite. The zeolite catalyst is preferably of MWW-type framework structure. The catalyst is thus preferably a “titanium zeolite of framework structure type MWW”, also referred to as “TiMWW”, which terms relate to a zeolite of framework structure MWW which contains titanium as isomorphous substitution element in the zeolitic framework. Preferably, the zeolitic framework is essentially free of aluminum and essentially consists of silicon, titanium, and oxygen.

The titanium containing zeolite preferably comprises one or more of Al, B, Zr, V, Nb, Ta, Cr, Mo, W, Mn, Fe, Co, Ni, Zn, Ga, Ge, In, Sn, Pb, Pd, Pt, Au, Cd, preferably one or more of B, Zr, V, Nb, Ta, Cr, Mo, W, Mn, Fe, Co, Ni, Zn, Ga, Ge, In, Sn, Pb, Pd, Pt, Au, Cd, more preferably Zn.

Preferably, the titanium zeolite of framework structure type MWW comprised in the catalyst contains titanium, calculated as elemental titanium, in an amount in the range of from 0.1 to 5 weight-%, more preferably from 0.2 to 4 weight-%, more preferably from 0.5 to 3 weight-%, more preferably from 1 to 2 weight-%, based on the total weight of the titanium zeolite of framework structure type MWW.

The feed stream subjected to epoxidation conditions in the reactor in the presence of the catalyst, and a reaction mixture comprising the propylene oxide and the organic solvent is obtained. The reactor can be operated in an isothermal or in an adiabatic manner, wherein it is preferred that the reactor is an isothermally operated reactor. Preferably, the reaction is carried out in a tube reactor or in a tube bundle reactor.

Preferably, the reaction temperature is in the range of from 20 to 100 °C , more preferably from 25 to 80 °C , more preferably from 25 to 60 °C , more preferably from 30 to 60 °C.

Preferably, the reaction pressure is in the range of from 5 to 100 bar, more preferably from 10 to 32 bar, more preferably from 15 to 25 bar, wherein the epoxidation reaction pressure is defined as the pressure at the exit of the isothermal reactor.

A product stream comprising the propylene oxide and the organic solvent is removed from the reactor. Said product stream is typically subjected to at least one work-up step to isolate the propylene oxide from the product stream. Further, the organic solvent, which typically comprises side products of the epoxidation reaction, is preferably subjected to one or more work-up steps to allow recirculation of the organic solvent, preferably acetonitrile, preferably after one or more purification steps, into step 1).

### Hydrolysis Reaction of Propylene Oxide to Produce Propylene Glycols

In one embodiment of this aspect, the propylene oxide is subjected to a hydrolysis reaction to produce propylene glycols.

The reaction proceeds without a catalyst and produces monopropylene glycol as main product along with dipropylene glycol, tripropylene glycol and polyglycols. Further details are given in Ullmann's Encyclopedia of Industrial Chemistry, 5^{th} ed., vol. A22, 239-252, 1993.

### Ring-Opening Polymerization of Propylene Oxide to Produce Polypropylene Glycols

In another embodiment of this aspect, the propylene oxide is subjected to a ring-opening polymerization to produce polypropylene glycols.

The ring-opening polymerization of propylene oxide is typically conducted via oxyanionic polymerization. The anionic polymerization is based on nucleophiles as initiators. The widely applied standard method for the technical synthesis of low molecular weight propylene gylcols is the controlled addition of propylene oxide to water or alcohols as initiators in the presence of alkaline catalysts. In most cases alkali metal compounds with high nucleophilicity are employed for this purpose. For higher molecular weights, alkali metal hydrides, alkyls, aryls, hydroxides, alkoxides, and amides can be employed for the living anionic polymerization of propylene oxide in an inert solvent.

The counterion should exhibit low Lewis acidity and preferentially little or no interaction with the chain end. Solvents employed for the anionic polymerization of epoxides must be polar and aprotic; therefore, tetrahydrofuran (THF), dioxane, dimethyl sulfoxide (DMSO), and hexamethylphosphoramide (HMPA) are often used. Alkoxides with sodium, potassium, or cesium counterions in ethers (most often THF) or other polar, aprotic solvents represent the most popular initiator systems. The addition of complexing agents, such as crown ethers suitable for the respective cation can strongly accelerate epoxide polymerization.

The oxyanionic polymerization of propylene oxide results in low molecular weight PPO with an unsaturated allyl end group. Due to this reaction, the molecular weight of PPO prepared by oxyanionic polymerization is limited to 6000 g/mol.

Further details with regard to the anionic polymerization of propylene oxide can be taken from Chem. Rev. 2016, 116, 2170 to 2243.

### Hydrogenation of COₓ From Epoxidation of Propylene

In one embodiment of this aspect, the epoxidation reaction progresses under formation of COₓ as a side product, and the process further comprises subjecting said COₓ to hydrogenation to produce at least one of synthesis gas, methanol, formaldehyde and formic acid, as described above with regard to the oxidation of propylene to acrylic acid.

1.-8. (canceled)

9. A process for the manufacture of an acrylic ester selected from n-butyl acrylate and 2-ethylhexyl acrylate, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion, to obtain renewably-sourced propylene; the olefin-interconversion comprising (i) and (ii):
(i) ethylene dimerization to obtain n-butenes; and
(ii) metathesis reaction between n-butenes obtained according to (i) and ethylene to obtain propylene; and
c) subjecting the renewably-sourced propylene to a sequence of chemical conversions to obtain an acrylic ester selected from n-butyl acrylate and 2-ethylhexyl acrylate, the sequence of chemical conversions comprising α), β), δ) and ε) or α), γ), δ) and ε):
α) hydroformylation of the propylene to produce n-butyraldehyde;
β) hydrogenation reaction of the n-butyraldehyde obtained in α) to produce n-butanol;
γ) condensation of the n-butyraldehyde obtained in α) to produce 2-ethyl-3-hydroxyhexanal and subjecting the 2-ethyl-3-hydroxyhexanal to a hydrogenation reaction to produce 2-ethylhexanol;
δ) oxidation reaction of the propylene to produce acrylic acid;
ε) esterification reaction of the acrylic acid with one of n-butanol obtained in β) or 2-ethylhexanol obtained in γ) to produce an acrylic ester.

10. The process according to claim 9, comprising
blending the renewably-sourced ethylene with complementary ethylene prior to step b), the complementary ethylene not being obtained from renewably-sourced ethanol in accordance with step a); and/or
blending the renewably-sourced propylene with complementary propylene prior to step c), the complementary propylene not being obtained from renewably-sourced ethanol in accordance with steps a) and b); and/or
blending the renewably-sourced n-butenes with complementary n-butenes prior to step b)-(ii), the complementary n-butenes not being obtained from renewably-sourced ethanol in accordance with steps a) and b)-(i).

11. The process according to claim 9, wherein step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractioning the effluent to recover a stream consisting essentially of n-butenes, a stream consisting essentially of heavier olefins, and an optional ethylene stream; and
optionally subjecting the stream consisting essentially of heavier olefins to hydrogenation so as to obtain renewably-sourced naphtha.

12. The process according to claim 9, wherein the n-butenes are a mixed stream including 1-butene and 2-butenes, and wherein b)-(ii) comprises removal of 1-butene from the mixed stream to obtain a stream rich in 2-butenes, and subjecting the stream rich in 2-butenes to the metathesis reaction.

13. The process according to claim 9, wherein the n-butenes are a mixed stream including 1-butene and 2-butenes, and wherein b)-(ii) comprises
b)-(iia) subjecting the mixed stream to the metathesis reaction to obtain propylene and unreacted 1-butene;
b)-(iib) subjecting the unreacted 1-butene to double bond isomerization to obtain 2-butenes; and
b)-(iic) recycling the 2-butenes obtained in step b)-(iib) to step b)-(iia).

14. The process according to claim 9, wherein the n-butenes are a mixed stream including 1-butene and 2-butenes, and wherein b)-(ii) comprises passing the mixed stream through a metathesis/isomerization zone comprising both a metathesis catalyst and an isomerization catalyst.

15. The process according claim 9, wherein the propylene oxidation reaction progresses under formation of COₓ as a side product, and the process further comprises subjecting said COₓ to hydrogenation to produce at least one of synthesis gas, methanol, formaldehyde and formic acid.

16. The process for enhancing the environmental sustainability of an acrylic ester selected from n-butyl acrylate and 2-ethylhexyl acrylate by blending or replacing a fossil-derived propylene with a renewably-sourced propylene to obtain a sustainability-enhanced propylene and subjecting the sustainability-enhanced propylene to a sequence of chemical conversions to obtain the acrylic ester, wherein the renewably-sourced propylene is obtained by
a) subjecting a feedstock comprising a renewably-sourced ethanol to dehydration to produce the renewably-sourced ethylene stream; and
b) subjecting the renewably-sourced ethylene stream to an olefin-interconversion, to obtain the renewably-sourced propylene; the olefin-interconversion comprising (i) and (ii):
(i) ethylene-dimerization to obtain n-butenes; and
(ii) metathesis reaction between n-butenes obtained according to (i) and ethylene to obtain propylene;
the sequence of chemical conversions comprising α), β), δ) and ε) or α), γ), δ) and ε):
α) hydroformylation of the propylene to produce n-butyraldehyde;
β) hydrogenation reaction of the n-butyraldehyde obtained in α) to produce n-butanol;
γ) condensation of the n-butyraldehyde obtained in α) to produce 2-ethyl-3-hydroxyhexanal and subjecting the 2-ethyl-3-hydroxyhexanal to a hydrogenation reaction to produce 2-ethylhexanol;
δ) oxidation reaction of the propylene to produce acrylic acid;
ε) esterification reaction of the acrylic acid with one of n-butanol obtained in β) or 2-ethylhexanol obtained in γ) to produce an acrylic ester.
